(19) United States
(12) Patent Application Publication
(10) Pub. No.: US 2013/0260376 A1
(43) Pub. Date: Oct. 3, 2013

(54) Prediction of and Monitoring Cancer Therapy Response Based on Gene Expression Profiling

(71) Applicants: Piyush Gupta, Boston, MA (US); Tamer T. Onder, Cambridge, MA (US); Eric S. Lander, Cambridge, MA (US); Robert Weinberg, Brookline, MA (US); Sendurai Mani, Houston, TX (US)

(72) Inventors: Piyush Gupta, Boston, MA (US); Tamer T. Onder, Cambridge, MA (US); Eric S. Lander, Cambridge, MA (US); Robert Weinberg, Brookline, MA (US); Sendurai Mani, Houston, TX (US)

(73) Assignees: WHITEHEAD INSTITUTE FOR BIOMEDICAL RESEARCH, Cambridge, MA (US); THE BROAD INSTITUTE, INC., Cambridge, MA (US)

(21) Appl. No.: 13/813,150

(22) PCT Filed: Aug. 2, 2011
(86) PCT No.: PCT/US11/46325

§ 371 Date: Jun. 10, 2013

Related U.S. Application Data
(60) Provisional application No. 61/369,928, filed on Aug. 2, 2010.

(51) Int. Cl. C12Q 1/68

(52) U.S. Cl. CPC C12Q 1/6886
USPC 435/6.11; 435/6.13; 506/9

(57) ABSTRACT

The invention utilizes gene expression profiles in methods of predicting the likelihood that a patient's cancer will respond to standard-of-care therapy. Also provided are methods of identifying therapeutic agents that target cancer stem cells or epithelial cancers that have undergone an epithelial to mesenchymal transition using such gene expression profiles.

### RELATED APPLICATIONS

This application claims priority to U.S. Ser. No. 61/369,928, filed on Aug. 2, 2010, which is herein incorporated by reference in its entirety.

### FIELD OF THE INVENTION

This invention concerns gene sets relevant to the treatment of epithelial cancers, and methods for assigning treatment options to epithelial cancer patients based upon knowledge derived from gene expression studies of cancer tissue.

### BACKGROUND OF THE INVENTION

Previous work has shown that epithelial-to-mesenchymal transition (“EMT”) is associated with metastasis and cancer stem cells (Creighton et al., 2009; Mani et al., 2008; Morel et al., 2008; Yang et al., 2006; Yang et al., 2004; Yauch et al., 2005) Importantly, induction of EMT across epithelial cancer types (e.g., lung, breast) also results in resistance to cancer therapies, including chemotherapies and kinase-targeted anti-cancer agents (e.g., erlotinib). Those skilled in the art will recognize that the EMT produces cancer cells that are invasive, migratory, and have stem-cell characteristics, which are all hallmarks of cells that have the potential to generate metastases.

EMT is a process in which adherent epithelial cells shed their epithelial characteristics and acquire, in their stead, mesenchymal properties, including fibroblastoid morphology, characteristic gene expression changes, increased potential for motility, and in the case of cancer cells, increased invasion, metastasis and resistance to chemotherapy. (See Kalluri et al., J Clin Invest 119(6):1420-28 (2009); Gupta et al., Cell 138(4):645-59 (2009)). Recent studies have linked EMTs with both metastatic progression of cancer (see Yang et al., Cell 117(7):927-39 (2004); Frixen et al., J Cell Biol 113(1):173-85 (1991); Sabbah et al., Drug Resist Updat 11(4-5):123-51 (2008)) and acquisition of stem-cell characteristics (see Mani et al., Cell 133(4):704-15 (2008); Morel et al., PLoS One 3(8):e288 (2008)), leading to the hypothesis that cancer cells that undergo an EMT are capable of metastasizing through their acquired invasiveness and, following dissemination, through their acquired self-renewal potential; the latter trait enables them to spawn the large cell populations that constitute macroscopic metastases.

Given these observations, one might predict that cancers harboring significant populations (or subpopulations) of cells having undergone EMT would be likely to exhibit reduced responsiveness to chemotherapies and anti-kinase targeted therapies.

### SUMMARY OF THE INVENTION

The present invention is a method for deriving a molecular signature of epithelial cancers that would not be responsive to chemotherapies and anti-kinase targeted therapies. The present invention also covers any patient stratification scheme that takes advantage of the biomarkers described herein, whether for the purpose of treatment selection and/or prognosis determination. Treatment selection could be either positive or negative and with respect to any class of anti-cancer agents. The method utilizes assays for the expression of biomarker genes that are upregulated in cancer cells post-EMT (Table 1) and assays for other biomarker genes upregulated in cells that have not undergone EMT (Table 2). Using these biomarker assays, it is possible to identify cancers that would not be responsive to conventional cancer therapies.

The invention provides methods of predicting the likelihood that a patient's epithelial cancer will respond to a standard-of-care therapy, following surgical removal of the primary tumor, by determining the expression level in cancer (i.e., in an epithelial cancer cell from the removed primary tumor) of genes in Tables 1 and/or 2, wherein the overexpression of genes in Table 1 indicates an increased likelihood that the tumor will be resistant to the standard-of-care therapy and overexpression of genes in Table 2 indicates an increased likelihood that the tumor will be sensitive to the standard-of-care therapy.

Overexpression of genes in Table 1 (or any suitable subset thereof) indicates an increased likelihood that the epithelial cancer will be resistant to standard-of-care therapies such as paclitaxel but sensitive to a cancer stem-cell selective agent (“CSS agent”) such as, for example, but not limited to, salinomycin. Moreover, underexpression of genes in Table 2 (or any suitable subset thereof) indicates an increased likelihood that the epithelial cancer will be resistant to standard-of-care therapy such as paclitaxel but sensitive to a CSS agent such as salinomycin.

Additionally, those skilled in the art will recognize that the underexpression of genes in Table 1 indicates an increased likelihood that the tumor will be sensitive to standard-of-care. Similarly, the overexpression of genes in Table 2 indicates an increased likelihood that the tumor will be resistant to standard-of-care therapy.

Those skilled in the art will recognize that determining the expression level of genes in Tables 1 and/or 2 occurs in vitro in the removed primary tumor.

Specifically, those skilled in the art will recognize that the overexpression of genes in Table 1 indicates an increased likelihood that the tumor will be resistant to standard-of-care therapy. For example, the overexpression of genes in Table 1 indicates an increased likelihood that the tumor will be resistant to paclitaxel.

Examples of standard-of-care therapy can include, but are not limited to, kinase-targeted therapy, such as EGFR-inhibition, radiation, a hormonal therapy, paclitaxel and/or any combination(s) thereof.

In various embodiments, those skilled in the art will recognize that the expression level of the genes assayed may constitute any subset of the genes in Table 1 and/or Table 2. Specifically, the gene subset is any subset of genes is one for which an appropriate statistical test (i.e., Gene Set Enrichment Analysis (“GSEA”)) demonstrates that the genes in the subset are differentially expressed in populations treated with a cancer therapy at a level of significance (e.g. p-value) less than 0.1, relative to an appropriate control population (e.g., DMSO treatment). Any appropriate statistical test(s) known to those skilled in the art and/or any appropriate control population(s) known to those skilled in the art can be used in identifying the gene subsets. For example, the appropriate control population(s) can be any population of cells (i.e., cancer cells) that have not been treated with a given cancer therapy.

Examples of cancer therapy may include, but are not limited to, salinomycin treatment and paclitaxel treatment. Moreover, in various embodiments, the subset of genes may include 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 1 and/or Table 2.

The overexpression of genes in Table 1 may also indicate an increased likelihood that the tumor will be sensitive to therapeutic agents that are toxic to cancer cells resistant to standard-of-care therapies. Moreover, the overexpression of genes in Table 1 may also indicate an increased likelihood that the tumor will be sensitive to therapeutic agents that are toxic to cancer stem cells or to therapeutic agents that target invasive and/or metastatic cancer cells. In still other embodiments, the overexpression of genes in Table 1 may indicate an increased likelihood that the tumor will be sensitive to therapeutic agents that are toxic to cancer cells that have undergone an epithelial-to-mesenchymal transition. Moreover, the overexpression of genes in Table 1 also indicates an increased likelihood that the tumor will be sensitive to a CSS agent (e.g., salinomycin).

Also provided are methods of predicting the likelihood that a patient's epithelial cancer will respond to standard-of-care therapy, following surgical removal of the primary tumor, comprising determining the expression level in cancer (i.e., in an epithelial cancer cell from the removed tumor) of genes in Table 2. Those skilled in the art will recognize that the reduced expression of genes in Table 2 indicates an increased likelihood that the tumor will be resistant to standard-of-care therapy. Standard-of-care therapy can include, but is not limited to, a kinase-targeted therapy, such as EGFR-inhibition; a radiation therapy; a hormonal therapy; paclitaxel; and/or any combination(s) thereof.

Those skilled in the art will recognize that determining the expression level of genes in Table 2 occurs in vitro in the removed primary tumor. Again, those skilled in the art will recognize that the expression level of the genes assayed may constitute any subset of the genes in Table 2. Specifically, the gene subset is any subset of genes is one for which an appropriate statistical test (i.e., Gene Set Enrichment Analysis (“GSEA”)) demonstrates that the genes in the subset are differentially expressed in populations treated with a cancer therapy at a level of significance (e.g. p-value) less than 0.1, relative to an appropriate control population (e.g., DMSO treatment). Any appropriate statistical test(s) known to those skilled in the art and/or any appropriate control population(s) known to those skilled in the art can be used in identifying the gene subsets. For example, the appropriate control population(s) can be any population of cells (i.e., cancer cells) that have not been treated with a given cancer therapy.

Examples of cancer therapy may include, but are not limited to, salinomycin treatment and paclitaxel treatment. Moreover, in various embodiments, the subset of genes may include 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 2.

In these methods, the reduced expression of genes in Table 2 may indicate an increased likelihood that the tumor will be sensitive to therapeutic agents that are toxic to cancer cells resistant to standard-of-care therapies. Similarly, the reduced expression of genes in Table 2 may indicate an increased likelihood that the tumor will be sensitive to therapeutic agents that are toxic to cancer stem cells. Likewise, the reduced expression of genes in Table 2 may indicate an increased likelihood that the tumor will be sensitive to therapeutic agents that are toxic to cancer cells that have undergone an epithelial-to-mesenchymal transition.

The invention further provides methods of identifying therapeutic agents that target cancer stem cells or epithelial cancers that have undergone an epithelial to mesenchymal transition by screening candidate agents to identify those that increase the levels of expression of the genes in Table 2, wherein an increase in the expression of genes in Table 2 indicates that the candidate agent targets cancer stem cells or epithelial cancers that have undergone an epithelial to mesenchymal transition. Moreover, the reduced expression of genes in Table 2 also indicates an increased likelihood that the tumor will be sensitive to a CSS agent (e.g., salinomycin).

Such methods are preferably performed in vitro on cancer (i.e., on epithelial cancer cells obtained following surgical removal of a primary tumor).

The methods of identifying therapeutic agents that target cancer stem cells or epithelial cancers that have undergone an EMT according to the invention can be performed independently, simultaneously, or sequentially.

Those skilled in the art will recognize that in these screening methods, any subset of genes in Table 2 is evaluated for its expression levels. Preferably, the subset of genes is one for which a statistical test demonstrates that the genes in the subset are differentially expressed in populations treated with a cancer therapy (e.g., salinomycin treatment or paclitaxel treatment) at a level of significance (e.g., p-value) less than 0.1, relative to an appropriate control population (e.g., DMSO treatment). For example, the subset of genes may include 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 2.

Any appropriate statistical test(s) known to those skilled in the art and/or any appropriate control population(s) known to those skilled in the art can be used in identifying the gene subsets. For example, the appropriate control population(s) can be any population of cells (i.e., cancer cells) that have not been treated with a given cancer therapy.

In still further embodiments, the invention provides methods of identifying therapeutic agents that target cancer stem cells or epithelial cancers that have undergone an epithelial to mesenchymal transition comprising screening candidate agents to identify those that decrease the levels of expression of the genes in Table 1, wherein a decrease in the expression of genes in Table 1 indicates that the candidate agent targets cancer stem cells or epithelial cancers that have undergone an epithelial to mesenchymal transition. Such methods are preferably performed in vitro on cancer (i.e., epithelial cancer cells obtained following surgical removal of a primary tumor).

In these methods, any subset of genes in Table 1 is evaluated for its expression levels. Preferably, the subset of genes is one for which a statistical test demonstrates that the genes in the subset are differentially expressed in populations treated with a cancer therapy (e.g., salinomycin treatment or paclitaxel treatment) at a level of significance (e.g., p-value) less than 0.1, relative to an appropriate control population (e.g., DMSO treatment). For example, the subset of genes may include 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 1.

Any appropriate statistical test(s) known to those skilled in the art and/or any appropriate control population(s) known to those skilled in the art can be used in identifying the gene subsets. For example, the appropriate control population(s) can be any population of cells (i.e., cancer cells) that have not been treated with a given cancer therapy.

In other embodiments, the invention provides methods of predicting the likelihood that a patient's epithelial cancer will respond to therapy, following surgical removal of the primary tumor, comprising determining the expression level in cancer of genes in Table 1. Those skilled in the art will recognize that the overexpression of genes in Table 1 indicates an increased likelihood that the tumor will be sensitive to therapy with salinomycin or other CSS agents. Moreover, the overexpression of genes in Table 1 indicates an increased likelihood that the tumor will be resistant to standard-of-care therapy such as, for example, paclitaxel.

Those skilled in the art will recognize that in such methods, determining the expression level of genes in Table 1 occurs in vitro in the removed primary tumor. In any of these methods of predicting the likelihood that a patient's epithelial cancer will respond to therapy, any subset of genes in Table 1 is evaluated for its expression levels. Preferably, the subset of the genes whose expression is evaluated is one for which a statistical test demonstrates that the genes in the subset are differentially expressed in populations treated with a cancer therapy (e.g., salinomycin treatment or paclitaxel treatment) at a level of significance (e.g., p-value) less than 0.1, relative to an appropriate control population (e.g., DMSO treatment). Those skilled in the art will recognize that the subset of genes can include 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 1.

Those skilled in the art will readily recognize that any appropriate statistical test(s) known to those skilled in the art and/or any appropriate control population(s) known to those skilled in the art can be used in identifying the gene subsets. For example, the appropriate control population(s) can be any population of cells (i.e., cancer cells) that have not been treated with a given cancer therapy.

In some embodiments, the methods of the invention provide intermediate information that may be useful to a skilled practitioner in selecting a future course of action, therapy, and/or treatment in a patient. For example, any of the methods described herein can further involve the step(s) of summarizing the data obtained by the determination of the gene expression levels. By way of non-limiting example, the summarizing may include prediction of the likelihood of long term survival of said patient without recurrence of the cancer following surgical removal of the primary tumor. Additionally (or alternatively), the summarizing may include recommendation for a treatment modality of said patient.

Also provided by the instant invention are kits containing, in one or more containers, at least one detectably labeled reagent that specifically recognizes one or more of the genes in Table 1 and/or Table 2. For example, the kits can be used to determine the level of expression of the one or more genes in Table 1 and/or Table 2 in cancer (i.e., in an epithelial cancer cell). In some embodiments, the kit is used to generate a biomarker profile of an epithelial cancer. Kits according to the invention can also contain at least one pharmaceutical excipient, diluent, adjuvant, or any combination(s) thereof.

Moreover, in any of the methods of the invention, the RNA expression levels are indirectly evaluated by determining protein expression levels of the corresponding gene products. For example, in one embodiment, the RNA expression levels are indirectly evaluated by determining chromatin states of the corresponding genes.

Those skilled in the art will readily recognize that the RNA is isolated from a fixed, wax-embedded breast cancer tissue specimen of said patient; the RNA is fragmented RNA; and/or the RNA is isolated from a fine needle biopsy sample.

In any of the methods described herein, the cancer may be an epithelial cancer, a lung cancer, breast cancer, prostate cancer, gastric cancer, colon cancer, pancreatic cancer, brain cancer, and/or melanoma cancer.

The invention additionally provides in vitro for determining whether or predicting the likelihood that a patient's epithelial cancer will respond to a standard-of-care therapy. Such methods involve the steps of determining the expression level in cancer (i.e., in an epithelial cancer cell obtained following surgical removal of a primary tumor from a patient having epithelial cancer) of genes in Tables 1 and/or 2, wherein the overexpression of genes in Table 1 indicates an increased likelihood that the patient's epithelial cancer will be resistant to the standard-of-care therapy and overexpression of genes in Table 2 indicates an increased likelihood that the patient's epithelial cancer will be sensitive to the standard-of-care therapy. More specifically, the overexpression of genes in Table 1 indicates an increased likelihood that the tumor will be resistant to standard-of-care therapy and/or an increased likelihood that the tumor will be resistant to paclitaxel. Moreover, the overexpression of genes in Table 1 indicates an increased likelihood that the tumor will be sensitive to therapeutic agents that are toxic to cancer cells resistant to standard-of-care therapies; an increased likelihood that the tumor will be sensitive to therapeutic agents that are toxic to cancer stem cells or to therapeutic agents that target invasive, metastatic, or invasive and metastatic cancer cells; and/or an increased likelihood that the tumor will be sensitive to therapeutic agents that are toxic to cancer cells that have undergone an epithelial-to-mesenchymal transition.

Similarly, the reduced expression of genes in Table 2 indicates an increased likelihood that the tumor will be resistant to standard-of-care therapy; an increased likelihood that the tumor will be sensitive to therapeutic agents that are toxic to cancer cells resistant to standard-of-care therapies; an increased likelihood that the tumor will be sensitive to therapeutic agents that are toxic to cancer stem cells; and/or an increased likelihood that the tumor will be sensitive to therapeutic agents that are toxic to cancer cells that have undergone an epithelial-to-mesenchymal transition.

Those skilled in the art will readily recognize that the standard-of-care therapy can be a kinase-targeted therapy, such as EGFR-inhibition; a radiation; a hormonal therapy; paclitaxel; and/or any combination thereof.

In any of these in vitro methods, the expression level of the genes assayed constitutes any subset of the genes in Table 1 and/or Table 2. Specifically, the subset of genes is one for which a statistical test (e.g., Gene Set Enrichment Analysis) demonstrates that the genes in the subset are differentially expressed in populations treated with a cancer therapy at a level of significance (e.g., p-value) less than 0.1, relative to an appropriate control population (e.g., DMSO treatment). Examples of cancer therapy include, but are not limited to salinomycin treatment and paclitaxel treatment. Those skilled in the art will recognize that the subset of genes assayed can include 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 1 and/or Table 2.

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural references unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference in their entirety.

### BRIEF DESCRIPTION OF THE FIGURES

FIG. 1: Heatmap summary of gene expression data from cells cultured in triplicate expressing one of five EMT-inducing factors (Goosecoid, TGFb, Snail, Twist or shRNA against E-cadherin) or expressing two control vectors (pWZL, shRNA against GFP). The legend depicts relative gene expression on a Log scale (base 2).

FIG. 2: Gene-set enrichment analysis using subsets of genes in Table 1. Shown is the enrichment level of subsets of EMT-associated genes in HMLER cancer cells treated with paclitaxel. The gene sets are named EMT_UP_NUM, where NUM is the number of genes in the subset. The plots show the enrichment score as a function of rank and indicate that each of the EMT_UP gene sets is enriched in its expression in cells following paclitaxel treatment.

FIG. 3: Gene-set enrichment analysis with subsets of genes in Table 2. Shown is the enrichment level of subsets of non-EMT-associated genes in HMLER cancer cells treated with paclitaxel. The gene sets are named EMT_DN_NUM, where NUM is the number of genes in the subset. The plots show the enrichment score as a function of rank and indicate that each of the EMT_DN gene sets is enriched in its expression in cells that are treated with DMSO control relative to cells treated with paclitaxel.

FIG. 4: Gene-set enrichment analysis with subsets of genes in Table 2. Shown is the enrichment level of subsets of non-EMT-associated genes in HMLER cancer cells treated with salinomycin. The gene sets are named EMT_DN_NUM, where NUM is the number of genes in the subset. The plots show the enrichment score as a function of rank and indicate that each of the EMT_DN gene sets is enriched in its expression in cells following salinomycin treatment relative to control treatment.

FIG. 5: Gene-set enrichment analysis with subsets of genes in Table 1. Shown is the enrichment level of subsets of EMT-associated genes in HMLER cancer cells treated with salinomycin. The gene sets are named EMT_UP_NUM, where NUM is the number of genes in the subset. The plots show the enrichment score as a function of rank and indicate that each of the EMT_UP gene sets is enriched in its expression in cells that are treated with DMSO control relative to cells treated with salinomycin.

### DETAILED DESCRIPTION OF THE INVENTION

Prior to setting forth the invention, it may be helpful to an understanding thereof to set forth definitions of certain terms that will be used hereinafter.

A “biomarker” in the context of the present invention is a molecular indicator of a specific biological property; a biochemical feature or facet that can be used to detect and/or categorize an epithelial cancer. “Biomarker” encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. In the instant invention, measurement of mRNA is preferred.

A “biological sample” or “sample” in the context of the present invention is a biological sample isolated from a subject and can include, by way of example and not limitation, whole blood, blood fraction, serum, plasma, blood cells, tissue biopsies, a cellular extract, a muscle or tissue sample, a muscle or tissue biopsy, or any other secretion, excretion, or other bodily fluids.

The phrase “differentially expressed” refers to differences in the quantity and/or the frequency of a biomarker present in a sample taken from patients having for example, epithelial cancer as compared to a control subject. For example without limitation, a biomarker can be an mRNA or a polypeptide which is present at an elevated level (i.e., overexpressed) or at a decreased level (i.e., underexpressed) in samples of patients with cancer as compared to samples of control subjects. Alternatively, a biomarker can be a polypeptide which is detected at a higher frequency (i.e., overexpressed) or at a lower frequency (i.e., underexpressed) in samples of patients compared to samples of control subjects. A biomarker can be differentially present in terms of quantity, frequency or both.

Previous work has shown that agents that selectively target cells induced into EMT also selectively kill cancer stem cells. Since cancer cells induced into EMT are also highly invasive, the hypothesis is that anti-cancer therapies that target invasive and/or metastatic cancer cells are likely to also target cancer cells induced into EMT.

According to one embodiment, this invention provides a method for determining which patient subpopulations harbor tumors responsive to three classes of essentially overlapping anti-cancer therapies or treatments—i.e., (a) therapies that target invasive/metastatic cells, (b) therapies that target cancer stem cells and (c) therapies that target cells post-EMT. Specifically, the invention provides methods for determining which therapies or treatments would be effective in cancers that express genetic biomarkers that are upregulated in cancer cells post-EMT (Table 1) and would not be effective in cancers that express genetic markers upregulated in cancer cells that have not undergone an EMT (Table 2).

The cancers that the methods of this invention are contemplated to be useful for include any epithelial cancers, and specifically include breast cancer, melanoma, brain, gastric, pancreatic cancer and carcinomas of the lung, prostate, and colon.

The anti-cancer therapies and treatments in which the methods of this invention are contemplated to be useful for include standard-of-care therapies such as paclitaxel, DNA damaging agents, kinase inhibitors (e.g., erlotinib), and radiation therapies, as well as therapies that target cancer stem cells and/or therapies that target cells post-EMT, including, for example, CSS agents such as salinomycin.

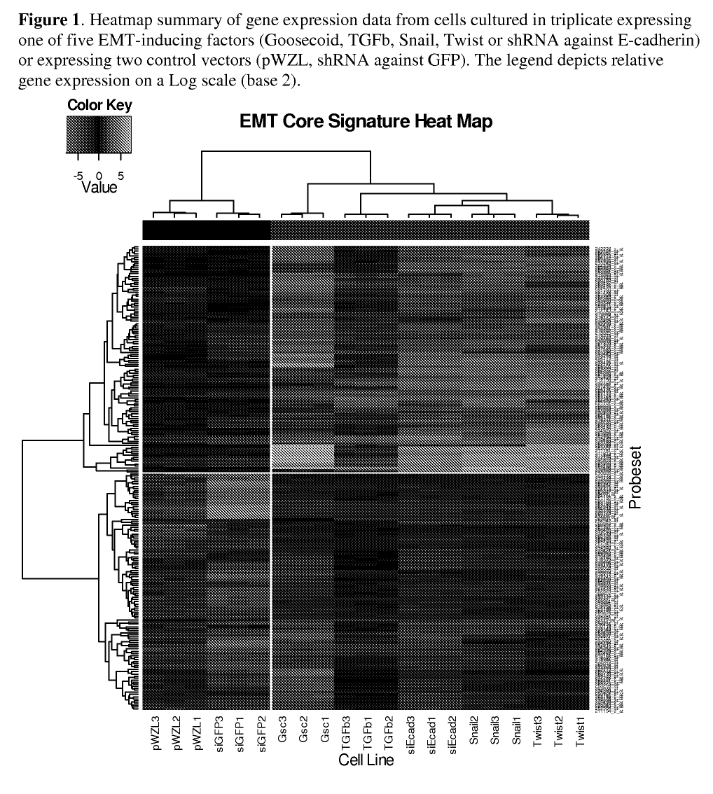

A set of genes differentially expressed in cancer cells that have undergone an EMT (Table 1) and genes expressed in cancer cells that have not undergone an EMT (Table 2) was determined. These genes were obtained by collecting RNA and performing microarray gene-expression analyses on breast cancer cells that were cultured either expressing one of 5 EMT-inducing genetic factors or 2 control genetic factors that did not induce EMT (control vectors). Cells were cultured in triplicate for each treatment condition. A global analysis of the gene expression data is shown as a heatmap in FIG. 1, where the top sets of genes in Tables 1 and 2 were used to construct the heatmap.

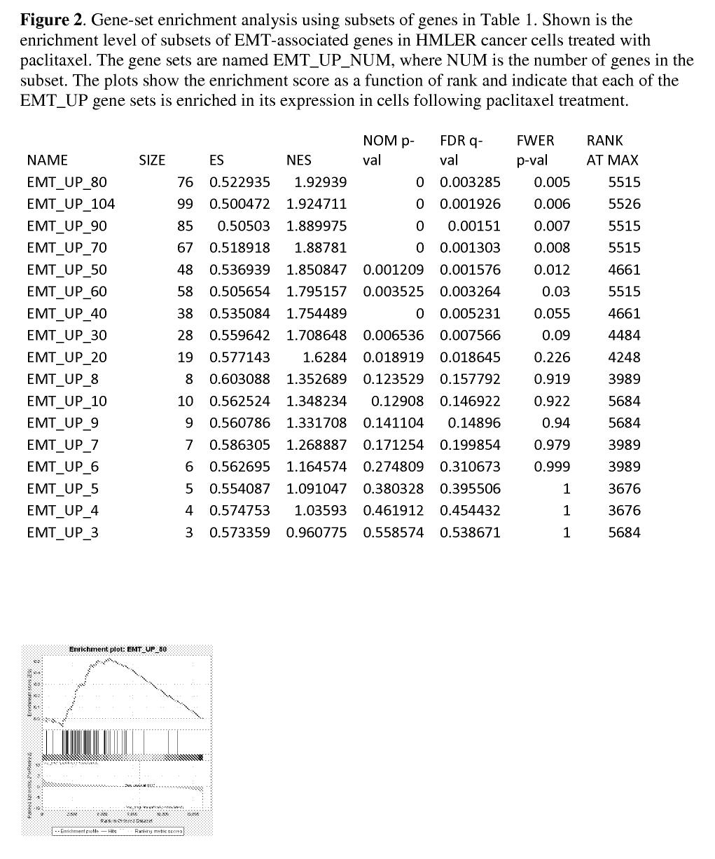

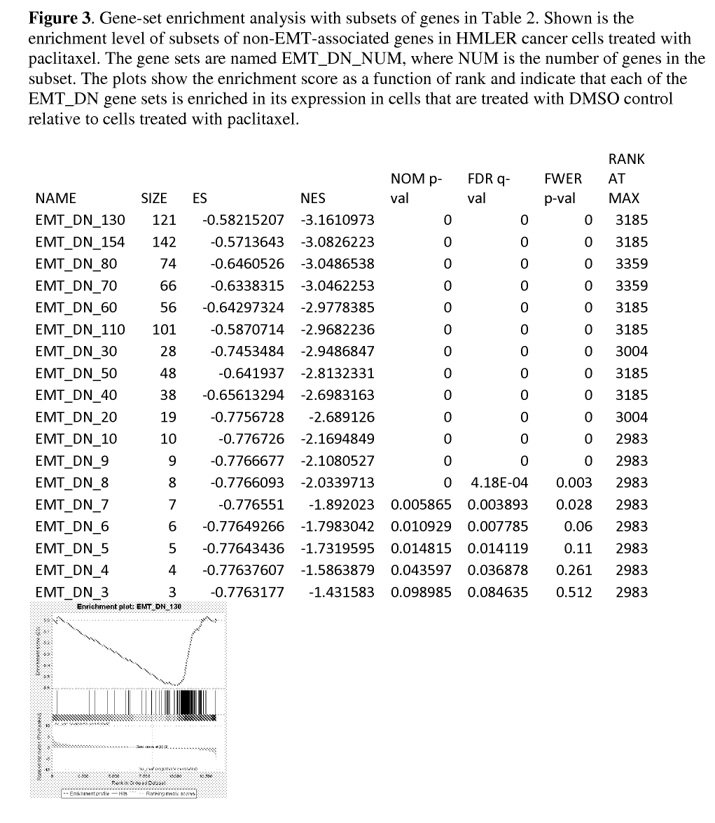

To demonstrate that the responsiveness of cancer cell populations to therapy can be both measured by and predicted by the various subsets of the genes identified in Tables 1 and 2, HMLER breast cancer populations were treated with a commonly used anti-cancer chemotherapy paclitaxel (Taxol) or with control DMSO treatment. mRNA was then isolated, and global gene expression data was collected. The collective expression levels of the genes in Tables 1 and 2 after paclitaxel treatment were then determined. For these analyses, which are shown in FIGS. 2 and 3, collections of gene subsets of various sizes were chosen.

Those skilled in the art will recognize that determining the expression level of genes in Tables 1 and/or 2 occurs in vitro in the removed primary tumor.

The analyses show that the genes expressed in Table 1 and/or many subsets thereof are over-expressed upon treatment with paclitaxel, indicating that these genes identify cancer cellular subpopulations that are resistant to treatment with paclitaxel. As a consequence, measurement of the expression of the genes in Table 1 would serve to identify tumors that would fail to be responsive to paclitaxel treatment when applied as a single agent.

Also covered in this invention is any subset of the genes in Table 1 for which a statistical test (such as, for example, Gene Set Enrichment Analysis (see Subramanian, Tamayo, et al., PNAS 102:15545-50 (2005) and Mootha, Lindgren et al., Nat. Genet 34:267-73 (2003), each of which is herein incorporated by reference in its entirety) demonstrates that the genes in the subset are over-expressed in paclitaxel-treated populations at a level of significance (e.g. p-value) less than 0.1, more preferably less than 0.05, relative to an appropriate control population (e.g., DMSO treatment). In one embodiment it was contemplated that the subset of genes from Table 1 comprises at least 2 genes, 10 genes, 15 genes, 20 genes or 30 genes (or any range intervening therebetween). For example, the subset might include 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes.

Those skilled in the art will recognize that any other appropriate statistical test(s) for gene enrichment or differential expression can also be used to identify the desired subset of genes from Table 1. For example, the summation of the log-transformed gene expression scores for the genes in a set could identify a metric that could be used to compare differential gene expression between two profiles using a t-test, modified t-test, or non-parametric test such as Mann-Whitney.

Moreover, those skilled in the art will also recognize that any appropriate control population(s) can also be used to identify the desired subset of genes from Table 1. For example, the appropriate control population(s) can be any population of cells (i.e., cancer cells) that have not been treated with a given cancer therapy.

Alternatively, the subsets of the genes in Table 1 may be identified as any subset for which a statistical test (such as, for example, Gene Set Enrichment Analysis) demonstrates that the genes in the subset are under-expressed in salinomycin-treated populations at a level of significance (e.g. p-value) less than 0.1, more preferably less that 0.05, relative to an appropriate control population (e.g., DMSO treatment). In one embodiment it was contemplated that the subset of genes from Table 1 comprises at least 2 genes, 10 genes, 15 genes, 20 genes or 30 genes (or any range intervening therebetween). For example, the subset might include 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes. For those skilled in the art, any other appropriate statistical test(s) for gene expression or differential expression can also be used to identify the desired subset of genes from Table 1. For example, the summation of the log-transformed gene expression scores for the genes in a set could identify a metric that could be used to compare differential gene expression between two profiles using a t-test, modified t-test, or non-parametric test such as Mann-Whitney.

Likewise, any appropriate control population(s) can also be used to identify the desired subset of genes from Table 1. For example, the appropriate control population(s) can be any population of cells (i.e., cancer cells) that have not been treated with a given cancer therapy.

Those skilled in the art will recognize that the statistical test used to determine suitable subsets of the genes in Table 1 could be Gene Set Enrichment Analysis (GSEA) (see Subramanian, Tamayo, et al., PNAS 102:15545-50 (2005) and Mootha, Lindgren et al., Nat. Genet 34:267-73 (2003), each of which is herein incorporated by reference in its entirety) as used for the purposes of elucidation in this application, or it could be any other statistical test of enrichment or expression known in the art. For example, the summation of the log-transformed gene expression scores for the genes in a set could identify a metric that could be used to compare differential gene expression between two profiles using a t-test, modified t-test, or non-parametric test such as Mann-Whitney.

The populations of cells being treated for the purposes of this evaluation could be cancer cells of any type or normal cellular populations.

TABLE 1
Genes identified that are over-expressed in cancer populations having undergone
an EMT, relative to cancer populations that have not undergone an EMT.

| | | | Mean Fold |
|---|---|---|---|
| | | | OverExpression |
| Symbol | Description | GenBank | Upon EMT |
| DCN | Decorin | AF138300 | 137.6156 |
| COL3A1 | collagen, type III, alpha 1 (Ehlers-Danlos | AU144167 | 132.1195 |
| | syndrome type IV, autosomal dominant) | | |
| COL1A2 | collagen, type I, alpha 2 | AA788711 | 88.05054 |
| FBN1 | fibrillin 1 (Marfan syndrome) | NM_000138 | 76.51337 |
| GREM1 | gremlin 1, cysteine knot superfamily, homolog | NM_013372 | 75.35859 |
| | (Xenopus laevis) | | |
| POSTN | periostin, osteoblast specific factor | D13665 | 73.18114 |
| NID1 | nidogen 1 | BF940043 | 51.91502 |
| FBLN5 | fibulin 5 | NM_006329 | 34.4268 |
| SDC2 | syndecan 2 (heparan sulfate proteoglycan 1, | AL577322 | 32.48001 |
| | cell surface-associated, fibroglycan) | | |
| COL5A2 | collagen, type V, alpha 2 | NM_000393 | 26.66545 |
| PRG1 | proteoglycan 1, secretory granule | J03223 | 23.46014 |
| TCF8 | transcription factor 8 (represses interleukin 2 | AI806174 | 22.83413 |
| | expression) | | |
| ENPP2 | ectonucleotide pyrophosphatase/ | L35594 | 22.72739 |
| | phosphodiesterase 2 (autotaxin) | | |
| NR2F1 | nuclear receptor subfamily 2, group F, member 1 | AI951185 | 20.64471 |
| COL6A1 | collagen, type VI, alpha 1 | AA292373 | 17.36271 |
| RGS4 | regulator of G-protein signalling 4 | AL514445 | 16.63788 |
| CDH11 | cadherin 11, type 2, OB-cadherin (osteoblast) | D21254 | 16.61483 |
| PRRX1 | paired related homeobox 1 | NM_006902 | 14.73362 |
| OLFML3 | olfactomedin-like 3 | NM_020190 | 14.0984 |
| | sparc/osteonectin, cwcv and kazal-like domains | | |
| SPOCK | proteoglycan (testican) | AF231124 | 13.99112 |
| | wingless-type MMTV integration site family, | | |
| WNT5A | member 5A | NM_003392 | 13.33384 |
| MAP1B | microtubule-associated protein 1B | AL523076 | 13.0877 |
| | | BG109855 | 12.44401 |
| PTX3 | pentraxin-related gene, rapidly induced by IL-1 | NM_002852 | 12.01196 |
| | beta | | |
| C5orf13 | chromosome 5 open reading frame 13 | U36189 | 11.95863 |
| IGFBP4 | insulin-like growth factor binding protein 4 | NM_001552 | 11.09963 |
| PCOLCE | procollagen C-endopeptidase enhancer | NM_002593 | 11.04575 |
| TNFAIP6 | tumor necrosis factor, alpha-induced protein 6 | NM_007115 | 11.02984 |
| LOC51334 | | NM_016644 | 10.91454 |
| CYP1B1 | cytochrome P450, family 1, subfamily B, | NM_000104 | 10.47429 |
| | polypeptide 1 | | |
| TFPI | tissue factor pathway inhibitor (lipoprotein- | BF511231 | 10.42648 |
| | associated coagulation inhibitor) | | |
| PVRL3 | poliovirus receptor-related 3 | AA129716 | 10.30262 |
| ROR1 | receptor tyrosine kinase-like orphan receptor 1 | NM_005012 | 10.10474 |
| FBLN1 | fibulin 1 | NM_006486 | 10.09844 |
| BIN1 | bridging integrator 1 | AF043899 | 9.928529 |
| LUM | Lumican | NM_002345 | 9.727574 |
| RGL1 | ral guanine nucleotide dissociation stimulator- | AF186779 | 9.643922 |
| | like 1 | | |
| PTGFR | prostaglandin F receptor (FP) | NM_000959 | 8.939536 |
| TGFBR3 | transforming growth factor, beta receptor III | NM_003243 | 8.838 |
| | (betaglycan, 300 kDa) | | |
| COL1A1 | collagen, type I, alpha 1 | Y15916 | 8.667645 |
| DLC1 | deleted in liver cancer 1 | AF026219 | 8.610518 |
| PMP22 | peripheral myelin protein 22 | L03203 | 8.560648 |
| PRKCA | protein kinase C, alpha | AI471375 | 8.338108 |
| MMP2 | matrix metallopeptidase 2 (gelatinase A, 72 kDa | NM_004530 | 8.268926 |
| | gelatinase, 72 kDa type IV collagenase) | | |
| CTGF | connective tissue growth factor | M92934 | 8.168776 |
| CDH2 | cadherin 2, type 1, N-cadherin (neuronal) | M34064 | 7.987921 |
| GNG11 | guanine nucleotide binding protein (G protein), | NM_004126 | 7.953115 |
| | gamma 11 | | |
| PPAP2B | phosphatidic acid phosphatase type 2B | AA628586 | 7.907272 |
| NEBL | Nebulette | AL157398 | 7.817894 |
| MYL9 | myosin, light polypeptide 9, regulatory | NM_006097 | 7.780485 |
| KCNMA1 | potassium large conductance calcium-activated | AI129381 | 7.747227 |
| | channel, subfamily M, alpha member 1 | | |
| IGFBP3 | insulin-like growth factor binding protein 3 | BF340228 | 7.57812 |
| CSPG2 | chondroitin sulfate proteoglycan 2 (versican) | NM_004385 | 7.318764 |
| SEMA5A | sema domain, seven thrombospondin repeats | NM_003966 | 7.298702 |
| | (type 1 and type 1-like), transmembrane domain | | |
| | (TM) and short cytoplasmic domain, | | |
| | (semaphorin) 5A | | |
| CITED2 | Cbp/p300-interacting transactivator, with | AF109161 | 7.220907 |
| | Glu/Asp-rich carboxy-terminal domain, 2 | | |
| MME | membrane metallo-endopeptidase (neutral | AI433463 | 7.05859 |
| | endopeptidase, enkephalinase, CALLA, CD10) | | |
| DOCK10 | dedicator of cytokinesis 10 | NM_017718 | 6.972809 |
| DNAJB4 | DnaJ (Hsp40) homolog, subfamily B, member 4 | BG252490 | 6.782043 |
| PCDH9 | protocadherin 9 | AI524125 | 6.711987 |
| NID2 | nidogen 2 (osteonidogen) | NM_007361 | 6.54739 |
| HAS2 | hyaluronan synthase 2 | NM_005328 | 6.520398 |
| PTGER4 | prostaglandin E receptor 4 (subtype EP4) | AA897516 | 6.396133 |
| TRAM2 | translocation associated membrane protein 2 | AI986461 | 6.275542 |
| SYT11 | synaptotagmin XI | BC004291 | 6.149546 |
| BGN | Biglycan | AA845258 | 5.838023 |
| CYBRD1 | cytochrome b reductase 1 | NM_024843 | 5.710828 |
| CHN1 | chimerin (chimaerin) 1 | BF339445 | 5.687127 |
| DPT | Dermatopontin | AI146848 | 5.573023 |
| ITGBL1 | integrin, beta-like 1 (with EGF-like repeat | AL359052 | 5.511939 |
| | domains) | | |
| FLJ22471 | | NM_025140 | 5.364784 |
| LOC221362 | | AL577024 | 5.35364 |
| MLPH | Melanophilin | NM_024101 | 5.296062 |
| ANXA6 | annexin A6 | NM_001155 | 5.18628 |
| EML1 | echinoderm microtubule associated protein like 1 | NM_004434 | 5.138332 |
| CREB3L1 | cAMP responsive element binding protein 3-like 1 | AF055009 | 5.073214 |
| FLJ10094 | | NM_017993 | 4.998863 |
| LRIG1 | leucine-rich repeats and immunoglobulin-like | AB050468 | 4.9963 |
| | domains 1 | | |
| SNED1 | sushi, nidogen and EGF-like domains 1 | N73970 | 4.993945 |
| SERPINF1 | serpin peptidase inhibitor, clade F (alpha-2 | NM_002615 | 4.969153 |
| | antiplasmin, pigment epithelium derived factor), | | |
| | member 1 | | |
| DAB2 | disabled homolog 2, mitogen-responsive | NM_001343 | 4.913939 |
| | phosphoprotein (Drosophila) | | |
| WASPIP | Wiskott-Aldrich syndrome protein interacting | AW058622 | 4.882974 |
| | protein | | |
| FN1 | fibronectin 1 | AJ276395 | 4.869319 |
| C10orf56 | chromosome 10 open reading frame 56 | AA131324 | 4.795629 |
| DAPK1 | death-associated protein kinase 1 | NM_004938 | 4.726984 |
| LOXL1 | lysyl oxidase-like 1 | NM_005576 | 4.720305 |
| ID2 | inhibitor of DNA binding 2, dominant negative | NM_002166 | 4.672064 |
| | helix-loop-helix protein | | |
| PTGER2 | prostaglandin E receptor 2 (subtype EP2), 53 kDa | NM_000956 | 4.427892 |
| COL8A1 | collagen, type VIII, alpha 1 | BE877796 | 4.38653 |
| DDR2 | discoidin domain receptor family, member 2 | NM_006182 | 4.338932 |
| SEPT6 | septin 6 | D50918 | 4.30699 |
| HRASLS3 | HRAS-like suppressor 3 | BC001387 | 4.281926 |
| PLEKHC1 | pleckstrin homology domain containing, family C | AW469573 | 4.272913 |
| | (with FERM domain) member 1 | | |
| THY1 | Thy-1 cell surface antigen | AA218868 | 4.253587 |
| RPS6KA2 | ribosomal protein S6 kinase, 90 kDa, | AI992251 | 4.225143 |
| | polypeptide 2 | | |
| GALC | galactosylceramidase (Krabbe disease) | NM_000153 | 4.222742 |
| FBN2 | fibrillin 2 (congenital contractural | NM_001999 | 4.205916 |
| | arachnodactyly) | | |
| FSTL1 | follistatin-like 1 | BC000055 | 4.175243 |
| NRP1 | neuropilin 1 | BE620457 | 4.162874 |
| TNS1 | tensin 1 | AL046979 | 4.131713 |
| TAGLN | Transgelin | NM_003186 | 4.131083 |
| CDKN2C | cyclin-dependent kinase inhibitor 2C (p18, | NM_001262 | 4.124788 |
| | inhibits CDK4) | | |
| MAGEH1 | melanoma antigen family H, 1 | NM_014061 | 4.094423 |
| LTBP2 | latent transforming growth factor beta binding | NM_000428 | 4.000998 |
| | protein 2 | | |
| PBX1 | pre-B-cell leukemia transcription factor 1 | AL049381 | 3.997339 |
| TBX3 | T-box 3 (ulnar mammary syndrome) | NM_016569 | 3.992244 |

The analyses also show that the genes in Table 2 and many subsets thereof are under-expressed upon treatment with paclitaxel, indicating that these genes identify cellular subpopulations that are sensitive to treatment with paclitaxel. As a consequence, measurement of the expression of the genes in Table 2 would serve to identify tumors that would be responsive to paclitaxel treatment when applied as a single agent.

Those skilled in the art will recognize that determining the expression level of genes in Table 2 occurs in vitro in the removed primary tumor.

Also covered in this invention is any subset of the genes in Table 2 for which a statistical test (such as, for example, Gene Set Enrichment Analysis) demonstrates that the genes in the subset are under-expressed in paclitaxel-treated populations at a level of significance (e.g. p-value) less than 0.1, more preferably less than 0.05, relative to an appropriate control population (e.g., DMSO treatment). In one embodiment it was contemplated that the subset of the genes from Table 2 comprises at least 2 genes, 6 genes, 10 genes, 15 genes, 20 genes or 30 genes (or any range intervening therebetween). For example, the subset might include 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes. Those skilled in the art will recognize that any other appropriate statistical test(s) for gene enrichment or differential expression can also be used to identify the desired subset of genes from Table 2. For example, the summation of the log-transformed gene expression scores for the genes in a set could identify a metric that could be used to compare differential gene expression between two profiles using a t-test, modified t-test, or non-parametric test such as Mann-Whitney.

Moreover, those skilled in the art will also recognize that any appropriate control population(s) can also be used to identify the desired subset of genes from Table 2. For example, the appropriate control population(s) can be any population of cells (i.e., cancer cells) that have not been treated with a given cancer therapy.

Alternatively, the subsets of the genes in Table 2 may be identified as any subset for which a statistical test (such as Gene Set Enrichment Analysis) demonstrates that the genes in the subset are over-expressed in salinomycin-treated populations at a level of significance (e.g. p-value) less than 0.1, more preferably less than 0.05, relative to an appropriate control population (e.g., DMSO treatment). In one embodiment it was contemplated that the subset of the genes from Table 2 comprises at least 2 genes, 6 genes, 10 genes, 15 genes, 20 genes or 30 genes (or any range intervening therebetween). For example, the subset might include 2, 3, 4, 5, 6, 7, 8, 9, 10, 11, 12, 13, 14, 15, 16, 17, 18, 19, 20, 21, 22, 23, 24, 25, 26, 27, 28, 29, or 30 genes. Those skilled in the art will recognize that any other appropriate statistical test(s) for gene enrichment or differential expression can also be used to identify can also be used to identify the desired subset of genes from Table 2. For example, the summation of the log-transformed gene expression scores for the genes in a set could identify a metric that could be used to compare differential gene expression between two profiles using a t-test, modified t-test, or non-parametric test such as Mann-Whitney.

Likewise, those skilled in the art will also recognize that any appropriate control population(s) can also be used to identify the desired subset of genes from Table 2. For example, the appropriate control population(s) can be any population of cells (i.e., cancer cells) that have not been treated with a given cancer therapy.

The statistical test used could be Gene Set Enrichment Analysis (GSEA) (see Subramanian, Tamayo, et al., PNAS 102:15545-50 (2005) and Mootha, Lindgren et al., Nat. Genet 34:267-73 (2003), each of which is herein incorporated by reference in its entirety) as used for the purposes of elucidation in this application, or it could be any other statistical test of enrichment or expression known in the art. By way of non-limiting example, the summation of the log-transformed gene expression scores for the genes in a set could identify a metric that could be used to compare differential gene expression between two profiles using a t-test, modified t-test, or non-parametric test such as Mann-Whitney.

The populations of cells being treated for the purposes of this evaluation could be cancer cells of any type or normal cellular populations.

TABLE 2
Genes identified that are over-expressed in cancer populations that have not
undergone an EMT, relative to cancer populations that have undergone an EMT.

| | | | Mean Fold |
|---|---|---|---|
| | | | OverExpression |
| Symbol | Description | GenBank | In Non-EMT |
| SERPINB2 | serpin peptidase inhibitor, clade B | NM_002575 | 36.74103 |
| | (ovalbumin), member 2 | | |
| TACSTD1 | tumor-associated calcium signal | NM_002354 | 35.91264 |
| | transducer 1 | | |
| SPRR1A | small proline-rich protein 1A | AI923984 | 34.99944 |
| SPRR1B | small proline-rich protein 1B (cornifin) | NM_003125 | 29.33599 |
| IL1A | interleukin 1, alpha | M15329 | 28.86922 |
| KLK10 | kallikrein 10 | BC002710 | 25.16523 |
| FGFR3 | fibroblast growth factor receptor 3 | NM_000142 | 24.74251 |
| | (achondroplasia, thanatophoric dwarfism) | | |
| CDH1 | cadherin 1, type 1, E-cadherin (epithelial) | NM_004360 | 23.74645 |
| SLPI | secretory leukocyte peptidase inhibitor | NM_003064 | 21.4404 |
| KRT6B | keratin 6B | AI831452 | 20.84833 |
| FXYD3 | FXYD domain containing ion transport | BC005238 | 19.01308 |
| | regulator 3 | | |
| PI3 | peptidase inhibitor 3, skin-derived | L10343 | 18.10103 |
| | (SKALP) | | |
| RAB25 | RAB25, member RAS oncogene family | NM_020387 | 17.64907 |
| SAA2 | serum amyloid A2 | M23699 | 17.20791 |
| RBM35A | RNA binding motif protein 35A | NM_017697 | 15.20696 |
| TMEM30B | transmembrane protein 30B | AV691491 | 14.98036 |
| EVA1 | epithelial V-like antigen 1 | AF275945 | 14.69364 |
| KLK7 | kallikrein 7 (chymotryptic, stratum corneum) | NM_005046 | 14.42981 |
| RBM35B | RNA binding motif protein 35A | NM_024939 | 13.49619 |
| S100A14 | S100 calcium binding protein A14 | NM_020672 | 13.44819 |
| SERPINB13 | serpin peptidase inhibitor, clade B | AJ001698 | 13.29747 |
| | (ovalbumin), member 13 | | |
| UCHL1 | ubiquitin carboxyl-terminal esterase L1 | NM_004181 | 13.27334 |
| | (ubiquitin thiolesterase) | | |
| ALDH1A3 | aldehyde dehydrogenase 1 family, | NM_000693 | 13.10531 |
| | member A3 | | |
| CKMT1B | creatine kinase, mitochondrial 1B | NM_020990 | 12.4713 |
| ANXA3 | annexin A3 | M63310 | 12.4013 |
| NMU | neuromedin U | NM_006681 | 12.15367 |
| KRT15 | keratin 15 | NM_002275 | 12.09266 |
| FST | Follistatin | NM_013409 | 11.85793 |
| FGFBP1 | fibroblast growth factor binding protein 1 | NM_005130 | 11.49472 |
| S100A7 | S100 calcium binding protein A7 | NM_002963 | 11.07673 |
| | (psoriasin 1) | | |
| TP73L | tumor protein p73-like | AF091627 | 10.93454 |
| FLJ12684 | | NM_024534 | 10.70372 |
| SCNN1A | sodium channel, nonvoltage-gated 1 alpha | NM_001038 | 10.3172 |
| KLK5 | kallikrein 5 | AF243527 | 10.20992 |
| S100A8 | S100 calcium binding protein A8 | NM_002964 | 10.10418 |
| | (calgranulin A) | | |
| CCND2 | cyclin D2 | AW026491 | 9.950438 |
| MAP7 | microtubule-associated protein 7 | AW242297 | 9.942027 |
| CXADR | coxsackie virus and adenovirus receptor | NM_001338 | 9.872805 |
| KRT17 | keratin 17 | NM_000422 | 9.74958 |
| CDH3 | cadherin 3, type 1, P-cadherin (placental) | NM_001793 | 9.735938 |
| TRIM29 | tripartite motif-containing 29 | NM_012101 | 9.373189 |
| SPINT1 | serine peptidase inhibitor, Kunitz type 1 | NM_003710 | 9.353589 |
| TGFA | transforming growth factor, alpha | NM_003236 | 9.30496 |
| IL18 | interleukin 18 (interferon-gamma-inducing | NM_001562 | 9.218934 |
| | factor) | | |
| CA9 | carbonic anhydrase IX | NM_001216 | 9.196596 |
| KRT16 | keratin 16 (focal non-epidermolytic | AF061812 | 9.177365 |
| | palmoplantar keratoderma) | | |
| GJB3 | gap junction protein, beta 3, 31 kDa | AF099730 | 9.030588 |
| | (connexin 31) | | |
| VSNL1 | visinin-like 1 | NM_003385 | 8.637896 |
| IL1B | interleukin 1, beta | NM_000576 | 8.629518 |
| CA2 | carbonic anhydrase II | M36532 | 8.606222 |
| CNTNAP2 | contactin associated protein-like 2 | AC005378 | 8.592036 |
| ARHGAP8 | Rho GTPase activating protein 8 | Z83838 | 8.434017 |
| KRT5 | keratin 5 (epidermolysis bullosa simplex, | NM_000424 | 8.14695 |
| | Dowling-Meara/Kobner/Weber-Cockayne types) | | |
| ARTN | Artemin | NM_003976 | 8.125857 |
| CAMK2B | calcium/calmodulin-dependent protein | AF078803 | 8.125181 |
| | kinase (CaM kinase) II beta | | |
| ZBED2 | zinc finger, BED-type containing 2 | NM_024508 | 8.046492 |
| TPD52L1 | tumor protein D52-like 1 | NM_003287 | 7.949147 |
| EPB41L4B | erythrocyte membrane protein band 4.1 | NM_019114 | 7.911 |
| | like 4B | | |
| KLK8 | kallikrein 8 (neuropsin/ovasin) | NM_007196 | 7.895551 |
| C1orf116 | chromosome 1 open reading frame 116 | NM_024115 | 7.889643 |
| LEPREL1 | leprecan-like 1 | NM_018192 | 7.85189 |
| JAG2 | jagged 2 | Y14330 | 7.562273 |
| DSC2 | desmocollin 2 | NM_004949 | 7.425664 |
| CYP27B1 | cytochrome P450, family 27, subfamily B, | NM_000785 | 7.293746 |
| | polypeptide 1 | | |
| HOOK1 | hook homolog 1 (Drosophila) | NM_015888 | 7.275468 |
| LGALS7 | lectin, galactoside-binding, soluble, 7 | NM_002307 | 7.241758 |
| | (galectin 7) | | |
| HBEGF | heparin-binding EGF-like growth factor | NM_001945 | 7.202511 |
| CDS1 | CDP-diacylglycerol synthase | NM_001263 | 7.130583 |
| | (phosphatidate cytidylyltransferase) 1 | | |
| RNF128 | ring finger protein 128 | NM_024539 | 7.12999 |
| PRR5 | | NM_015366 | 7.124753 |
| KRT6A | keratin 6A | J00269 | 7.042267 |
| LAMA3 | laminin, alpha 3 | NM_000227 | 6.95736 |
| AP1M2 | adaptor-related protein complex 1, mu 2 | NM_005498 | 6.911026 |
| | subunit | | |
| SLAC2-B | | AB014524 | 6.847038 |
| GRHL2 | grainyhead-like 2 (Drosophila) | NM_024915 | 6.781949 |
| ST14 | suppression of tumorigenicity 14 (colon | NM_021978 | 6.733796 |
| | carcinoma, matriptase, epithin) | | |
| DSC3 | desmocollin 3 | NM_001941 | 6.68478 |
| CD24 | CD24 antigen (small cell lung carcinoma | M58664 | 6.653991 |
| | cluster 4 antigen) | | |
| LAMB3 | laminin, beta 3 | L25541 | 6.6375 |
| TSPAN1 | tetraspanin 1 | AF133425 | 6.619673 |
| SYK | spleen tyrosine kinase | NM_003177 | 6.585623 |
| SNX10 | sorting nexin 10 | NM_013322 | 6.540949 |
| | | NM_024064 | 6.518229 |
| CTSL2 | cathepsin L2 | AF070448 | 6.516422 |
| SLC2A9 | solute carrier family 2 (facilitated glucose | NM_020041 | 6.458325 |
| | transporter), member 9 | | |
| TMEM40 | transmembrane protein 40 | NM_018306 | 6.408648 |
| COL17A1 | collagen, type XVII, alpha 1 | NM_000494 | 6.405184 |
| C10orf10 | chromosome 10 open reading frame 10 | AL136653 | 6.37754 |
| ST6GALNAC2 | ST6 (alpha-N-acetyl-neuraminyl-2,3-beta- | NM_006456 | 6.224336 |
| | galactosyl-1,3)-N-acetylgalactosaminide | | |
| | alpha-2,6-sialyltransferase 2 | | |
| ANXA8 | annexin A8 | NM_001630 | 6.199621 |
| ABLIM1 | actin binding LIM protein 1 | NM_006720 | 6.19859 |
| RLN2 | relaxin 2 | NM_005059 | 6.139665 |
| VGLL1 | vestigial like 1 (Drosophila) | BE542323 | 6.116473 |
| NRG1 | neuregulin 1 | NM_013959 | 5.854395 |
| MMP9 | matrix metallopeptidase 9 (gelatinase B, | NM_004994 | 5.737173 |
| | 92 kDa gelatinase, 92 kDa type IV | | |
| | collagenase) | | |
| DSG3 | desmoglein 3 (pemphigus vulgaris antigen) | NM_001944 | 5.731926 |
| GJB5 | gap junction protein, beta 5 (connexin 31.1) | NM_005268 | 5.684999 |
| NDRG1 | N-myc downstream regulated gene 1 | NM_006096 | 5.681532 |
| MAPK13 | mitogen-activated protein kinase 13 | BC000433 | 5.587721 |
| DST | Dystonin | NM_001723 | 5.560135 |
| CORO1A | coronin, actin binding protein, 1A | U34690 | 5.510182 |
| IRF6 | interferon regulatory factor 6 | AU144284 | 5.499117 |
| KIBRA | | AK001727 | 5.491803 |
| SPINT2 | serine peptidase inhibitor, Kunitz type, 2 | AF027205 | 5.466358 |
| ALOX15B | arachidonate 15-lipoxygenase, second type | NM_001141 | 5.461662 |
| SERPINB1 | serpin peptidase inhibitor, clade B | NM_030666 | 5.348966 |
| | (ovalbumin), member 1 | | |
| CLCA2 | chloride channel, calcium activated, family | AF043977 | 5.30091 |
| | member 2 | | |
| MYO5C | myosin VC | NM_018728 | 5.269624 |
| CSTA | cystatin A (stefin A) | NM_005213 | 5.215624 |
| ITGB4 | integrin, beta 4 | NM_000213 | 5.180603 |
| MBP | myelin basic protein | AW070431 | 5.108643 |
| AQP3 | aquaporin 3 | N74607 | 5.084832 |
| SLC7A5 | solute carrier family 7 (cationic amino acid | AB018009 | 5.084409 |
| | transporter, y+ system), member 5 | | |
| GPR87 | G protein-coupled receptor 87 | NM_023915 | 5.073566 |
| MALL | mal, T-cell differentiation protein-like | BC003179 | 4.957731 |
| MST1R | macrophage stimulating 1 receptor (c-met- | NM_002447 | 4.955876 |
| | related tyrosine kinase) | | |
| SOX15 | SRY (sex determining region Y)-box 15 | NM_006942 | 4.948873 |
| LAMC2 | laminin, gamma 2 | NM_005562 | 4.941675 |
| CST6 | cystatin E/M | NM_001323 | 4.931341 |
| MFAP5 | microfibrillar associated protein 5 | AW665892 | 4.871412 |
| KRT18 | keratin 18 | NM_000224 | 4.799686 |
| JUP | junction plakoglobin | NM_021991 | 4.719454 |
| DSP | Desmoplakin | NM_004415 | 4.716772 |
| MTSS1 | metastasis suppressor 1 | NM_014751 | 4.715399 |
| FGFR2 | fibroblast growth factor receptor 2 | NM_022969 | 4.67323 |
| | (bacteria-expressed kinase, keratinocyte | | |
| | growth factor receptor, craniofacial | | |
| | dysostosis 1, Crouzon syndrome, Pfeiffer | | |
| | syndrome, Jackson-Weiss syndrome) | | |
| PKP3 | plakophilin 3 | AF053719 | 4.646421 |
| STAC | SH3 and cysteine rich domain | NM_003149 | 4.643331 |
| RAB38 | RAB38, member RAS oncogene family | NM_022337 | 4.544243 |
| SFRP1 | secreted frizzled-related protein 1 | NM_003012 | 4.465928 |
| RHOD | ras homolog gene family, member D | BC001338 | 4.45418 |
| TPD52 | tumor protein D52 | BG389015 | 4.453563 |
| F11R | F11 receptor | AF154005 | 4.39018 |
| TNFRSF6B | tumor necrosis factor receptor | NM_003823 | 4.342302 |
| | superfamily, member 6b, decoy | | |
| BIK | BCL2-interacting killer (apoptosis- | NM_001197 | 4.323681 |
| | inducing) | | |
| XDH | xanthine dehydrogenase | U06117 | 4.309678 |
| PLA2G4A | phospholipase A2, group IVA (cytosolic, | M68874 | 4.308364 |
| | calcium-dependent) | | |
| PTHLH | parathyroid hormone-like hormone | J03580 | 4.294946 |
| NEF3 | neurofilament 3 (150 kDa medium) | NM_005382 | 4.274928 |
| SORL1 | sortilin-related receptor, L(DLR class) A | AV728268 | 4.257894 |
| | repeats-containing | | |
| SLC6A8 | solute carrier family 6 (neurotransmitter | NM_005629 | 4.205508 |
| | transporter, creatine), member 8 | | |
| PRRG4 | proline rich Gla (G-carboxyglutamic acid) | NM_024081 | 4.187822 |
| | 4 (transmembrane) | | |
| CLDN1 | claudin 1 | NM_021101 | 4.185384 |
| KIAA0888 | | AB020695 | 4.162009 |
| GPR56 | G protein-coupled receptor 56 | AL554008 | 4.153478 |
| SNCA | synuclein, alpha (non A4 component of | BG260394 | 4.149795 |
| | amyloid precursor) | | |
| FLRT3 | fibronectin leucine rich transmembrane | NM_013281 | 4.130167 |
| | protein 3 | | |
| IL1RN | interleukin 1 receptor antagonist | U65590 | 4.12988 |
| DDR1 | discoidin domain receptor family, member 1 | L11315 | 4.125646 |
| LYN | v-yes-1 Yamaguchi sarcoma viral related | M79321 | 4.107271 |
| | oncogene homolog | | |
| FLJ20130 | | NM_017681 | 4.09499 |
| STAP2 | | BC000795 | 4.089544 |
| KCNK1 | potassium channel, subfamily K, member 1 | NM_002245 | 4.084162 |
| TSPAN13 | tetraspanin 13 | NM_014399 | 4.079691 |
| LISCH7 | | NM_015925 | 4.025813 |
| PERP | PERP, TP53 apoptosis effector | NM_022121 | 4.024473 |

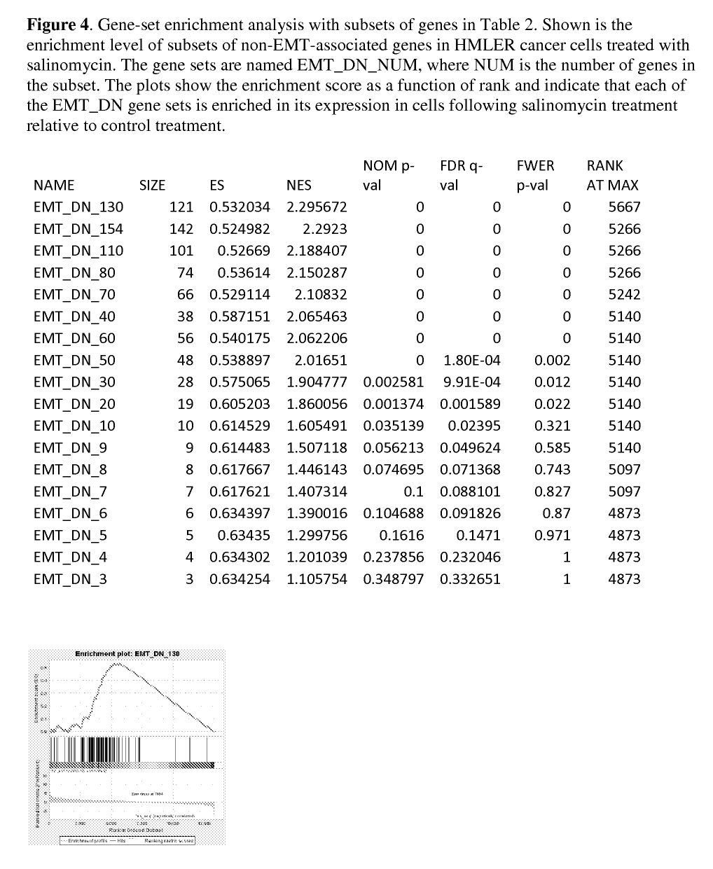

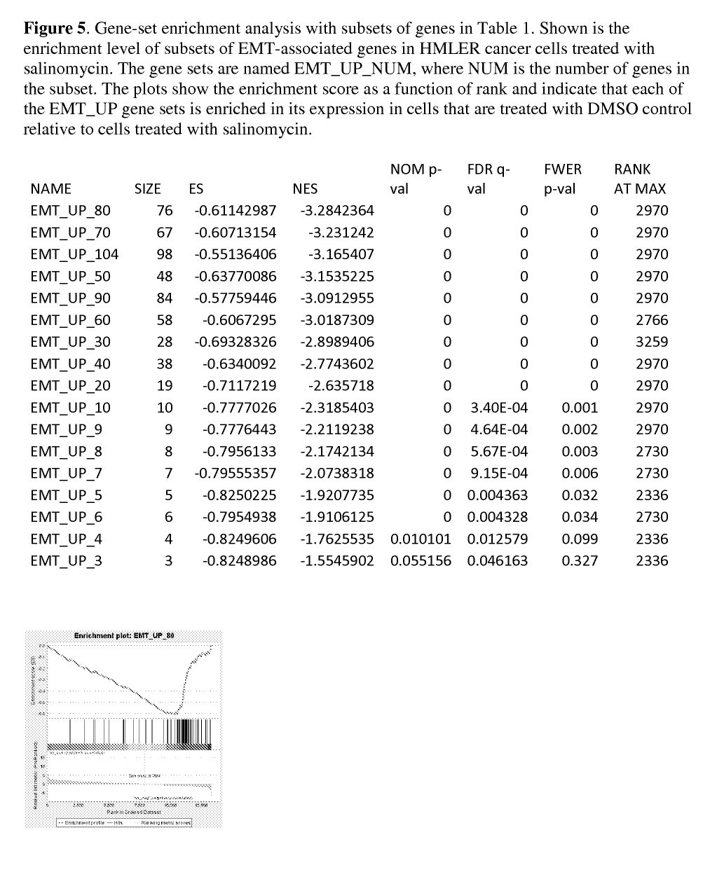

Next, identical analyses as those described above were performed in the context of treatment with a different anti-cancer agent—salinomycin—that was previously identified as specifically killing invasive cancer stem cells. The opposite expression change (relative to paclitaxel) was observed upon treatment with salinomycin. The analyses, shown in FIGS. 4 and 5, indicate that the genes expressed in Table 1 and any subsets thereof are under-expressed upon treatment with salinomycin, indicating that these genes identify cellular subpopulations that are sensitive to treatment with a CSS agent such as salinomycin. As a consequence, measurement of the expression of the genes in Table 1 (or any appropriate subsets thereof identified according to the methods disclosed herein) would serve to identify tumors that would be responsive to a CSS agent (e.g., salinomycin treatment) when applied as a single agent.

The analyses also show that the genes expressed in Table 2 and any subset thereof are over-expressed upon treatment with salinomycin (relative to control), indicating that these genes identify cellular subpopulations that are resistant to treatment with a CSS agent such as salinomycin. As a consequence, measurement of the expression of the genes in Table 2 (or any appropriate subsets thereof identified according to the methods disclosed herein) would serve to identify tumors that would fail to be responsive to a CSS agent (e.g., salinomycin treatment) when applied as a single agent.

It follows that measurement of the expression of the genes in Tables 1 and/or 2 as well as various subsets thereof for which a statistical test demonstrates that the genes in the subset are differentially expressed in response to treatment with a cancer treatment (e.g., salinomycin treatment or paclitaxel treatment) at a level of significance (e.g., p value) less than 0.1, relative to an appropriate control population (e.g., DMSO treatment) can be used to identify cancer cell populations that are or are not responsive to any given therapy or treatment. Distinct subpopulations of cells are identified using the expression levels of the genes in Tables 1 and/or 2 (or any appropriate subsets thereof) and these distinct subpopulations could respond distinctively to any particular therapeutic or treatment regimen, thereby allowing these genes to serve as biomarkers dictating therapy choice following primary tumor removal.

All documents and patents or patent applications referred to herein are fully incorporated by reference.

### REFERENCES

1. Piyush Gupta, Tamer T. Onder, Sendurai Mani, Mai-jing Liao, Eric S. Lander, Robert A. Weinberg. A Method for the Discovery of Agents Targeting and Exhibiting Specific Toxicity for Cancer Stem Cells. Patent pending. (WHI07-20; MIT 12947WB; WO/2009/126310).
2. Piyush B. Gupta, Tamer T. Onder, Guozhi Jiang, Tai Kao, Charlotte Kuperwasser, Robert A. Weinberg, Eric S. Lander. “Identification of selective inhibitors of cancer stem cells by high-throughput screening.” Cell. (2009) August; 138(4):645-659.
3. Thomson S, Petti F, Sujka-Kwok I, Epstein D, Haley J D. Kinase switching in mesenchymal-like non-small cell lung cancer lines contributes to EGFR inhibitor resistance through pathway redundancy. Clin Exp Metastasis. 2008; 25(8):843-54. Epub 2008 Aug. 12. PubMed PMID: 18696232.
4. Barr S, Thomson S, Buck E, Russo S, Petti F, Sujka-Kwok I, Eyzaguirre A, Rosenfeld-Franklin M, Gibson N W, Miglarese M, Epstein D, Iwata K K, Haley J D. Bypassing cellular EGF receptor dependence through epithelial-to-mesenchymal-like transitions. Clin Exp Metastasis. 2008; 25(6):685-93. Epub 2008 Jan. 31. Review. PubMed PMID: 18236164; PubMed Central PMCID: PMC2471394.
5. Buck E, Eyzaguirre A, Barr S, Thompson S, Sennello R, Young D, Iwata K K, Gibson N W, Cagnoni P, Haley J D. Loss of homotypic cell adhesion by epithelial-mesenchymal transition or mutation limits sensitivity to epidermal growth factor receptor inhibition. Mol Cancer Ther. 2007 February; 6(2):532-41. PubMed PMID: 17308052.
6. Woodward W A, Debeb B G, Xu W, Buchholz T A. Overcoming radiation resistance in inflammatory breast cancer. Cancer. 2010 Jun. 1; 116(11 Suppl):2840-5. PubMed PMID:20503417.
7. Bao, S., Wu, Q., McLendon, R. E., Hao, Y., Shi, Q., Hjelmeland, A. B., Dewhirst, M. W., Bigner, D. D., and Rich, J. N. (2006). Glioma stem cells promote radioresistance by preferential activation of the DNA damage response. Nature 444, 756-760.
8. Barr, S., Thomson, S., Buck, E., Russo, S., Petti, F., Sujka-Kwok, I., Eyzaguirre, A., Rosenfeld-Franklin, M., Gibson, N. W., Miglarese, M., et al. (2008). Bypassing cellular EGF receptor dependence through epithelial-to-mesenchymal-like transitions. Clinical & experimental metastasis 25, 685-693.
9. Buck, E., Eyzaguirre, A., Rosenfeld-Franklin, M., Thomson, S., Mulvihill, M., Barr, S., Brown, E., O'Connor, M., Yao, Y., Pachter, J., et al. (2008). Feedback mechanisms promote cooperativity for small molecule inhibitors of epidermal and insulin-like growth factor receptors. Cancer research 68, 8322-8332.
10. Creighton, C. J., Li, X., Landis, M., Dixon, J. M., Neumeister, V. M., Sjolund, A., Rimm, D. L., Wong, H., Rodriguez, A., Herschkowitz, J. I., et al. (2009). Residual breast cancers after conventional therapy display mesenchymal as well as tumor-initiating features. Proceedings of the National Academy of Sciences of the United States of America 106, 13820-13825.
11. Horwitz, K. B., and Sartorius, C. A. (2008). Progestins in hormone replacement therapies reactivate cancer stem cells in women with preexisting breast cancers: a hypothesis. The Journal of clinical endocrinology and metabolism 93, 3295-3298. Mani, S. A., Guo, W., Liao, M. J., Eaton, E. N., Ayyanan, A., Zhou, A. Y., Brooks, M., Reinhard, F., Zhang, C. C., Shipitsin, M., et al. (2008). The epithelial-mesenchymal transition generates cells with properties of stem cells. Cell 133, 704-715.
12. Morel, A. P., Lievre, M., Thomas, C., Hinkal, G., Ansieau, S., and Puisieux, A. (2008). Generation of breast cancer stem cells through epithelial-mesenchymal transition. PLoS ONE 3, e2888.
13. Thomson, S., Buck, E., Petti, F., Griffin, G., Brown, E., Ramnarine, N., Iwata, K. K., Gibson, N., and Haley, J. D. (2005). Epithelial to mesenchymal transition is a determinant of sensitivity of non-small-cell lung carcinoma cell lines and xenografts to epidermal growth factor receptor inhibition. Cancer research 65, 9455-9462.
14. Yang, A. D., Fan, F., Camp, E. R., van Buren, G., Liu, W., Somcio, R., Gray, M. J., Cheng, H., Hoff, P. M., and Ellis, L. M. (2006). Chronic oxaliplatin resistance induces epithelial-to-mesenchymal transition in colorectal cancer cell lines. Clin Cancer Res 12, 4147-4153.
15. Yang, J., Mani, S. A., Donaher, J. L., Ramaswamy, S., Itzykson, R. A., Come, C., Savagner, P., Gitelman, I., Richardson, A., and Weinberg, R. A. (2004). Twist, a master regulator of morphogenesis, plays an essential role in tumor metastasis. Cell 117, 927-939.
16. Yauch, R. L., Januario, T., Eberhard, D. A., Cavet, G., Zhu, W., Fu, L., Pham, T. Q., Soriano, R., Stinson, J., Seshagiri, S., et al. (2005). Epithelial versus mesenchymal phenotype determines in vitro sensitivity and predicts clinical activity of erlotinib in lung cancer patients. Clin Cancer Res 11, 8686-8698.
17. Taube, J. H, Herschkowitz, J. I., Komurov, K., Zhou, A. Y., Gupta, S., Yang, J., Hartwell, K., Onder, T. T., Gupta, P. B., Evans, K. W., Hollier, B. G., Ram, P. T., Lander, E. S., Rosen, J. M., Weinberg, R. A., Mani, S. A. (2010). A Core EMT Interactome Gene Expression Signature is Associated with Claudin-Low and Metaplastic Breast Cancer Subtypes. Proc. Natl Acad. Sci 107, 15449-15454.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of predicting the likelihood that a patient's epithelial cancer will respond to a standard-of-care therapy, following surgical removal of the primary tumor, comprising determining the expression level in cancer of genes in Tables 1 or 2, wherein the overexpression of genes in Table 1 indicates an increased likelihood that the tumor will be resistant to the standard-of-care therapy and overexpression of genes in Table 2 indicates an increased likelihood that the tumor will be sensitive to the standard-of-care therapy.

2. The method of claim 1, wherein the overexpression of genes in Table 1 indicates an increased likelihood that the tumor will be resistant to standard-of-care therapy.

3. The method of claim 2 wherein the overexpression of genes in Table 1 indicates an increased likelihood that the tumor will be resistant to paclitaxel.

4. The method of claim 1, wherein the standard-of-care therapy is a kinase-targeted therapy, such as EGFR-inhibition.

5. The method of claim 1, wherein the standard-of-care therapy is a radiation.

6. The method of claim 1, wherein the standard-of-care therapy is a hormonal therapy.

7. The method of claim 1, wherein the therapy is a combination of therapies indicated in claims 3-6.

8. The method of claim 1, wherein the expression level of the genes assayed constitutes any subset of the genes in Table 1 or Table 2.

9. The method of claim 8, wherein the subset of genes is one for which a statistical test demonstrates that the genes in the subset are differentially expressed in populations treated with a cancer therapy at a level of significance less than 0.1, relative to an appropriate control population.

10. The method of claim 9, wherein the cancer therapy is selected from the group consisting of salinomycin treatment and paclitaxel treatment.

11. The method of claim 8, wherein the subset of genes comprises 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 1 or Table 2.

12. The method of claim 1, wherein the overexpression of genes in Table 1 indicates an increased likelihood that the tumor will be sensitive to therapeutic agents that are toxic to cancer cells resistant to standard-of-care therapies.

13. The method of claim 1, wherein the overexpression of genes in Table 1 indicates an increased likelihood that the tumor will be sensitive to therapeutic agents that are toxic to cancer stem cells or to therapeutic agents that target invasive, metastatic, or invasive and metastatic cancer cells.

14. The method of claim 1, wherein the overexpression of genes in Table 1 indicates an increased likelihood that the tumor will be sensitive to therapeutic agents that are toxic to cancer cells that have undergone an epithelial-to-mesenchymal transition.

15. The method of claim 1, wherein the overexpression of genes in Table 1 indicates an increased likelihood that the tumor will be sensitive to salinomycin.

16. A method of predicting the likelihood that a patient's epithelial cancer will respond to standard-of-care therapy, following surgical removal of the primary tumor, comprising determining the expression level in cancer of genes in Table 2.

17. The method of claim 16, wherein the reduced expression of genes in Table 2 indicates an increased likelihood that the tumor will be resistant to standard-of-care therapy.

18. The method of claim 16, wherein the standard-of-care therapy is a kinase-targeted therapy, such as EGFR-inhibition.

19. The method of claim 16, wherein the standard-of-care therapy is a radiation therapy.

20. The method of claim 16, wherein the standard-of-care therapy is a hormonal therapy.

21. The method of claim 16, wherein the standard-of-care therapy is paclitaxel.

22. The method of claim 16, wherein the standard-of-care therapy is a combination of therapies indicated in claims 17-21.

23. The method of claim 16, wherein the expression level of the genes assayed constitutes any subset of the genes in Table 2.

24. The method of claim 23, wherein the subset of genes is one for which a statistical test demonstrates that the genes in the subset are differentially expressed in populations treated with a cancer therapy at a level of significance less than 0.1, relative to an appropriate control population.

25. The method of claim 24, wherein the cancer therapy is selected from the group consisting of salinomycin treatment and paclitaxel treatment.

26. The method of claim 23, wherein the subset of genes comprises 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 2.

27. The method of claim 16, wherein the reduced expression of genes in Table 2 indicates an increased likelihood that the tumor will be sensitive to therapeutic agents that are toxic to cancer cells resistant to standard-of-care therapies.

28. The method of claim 16, wherein the reduced expression of genes in Table 2 indicates an increased likelihood that the tumor will be sensitive to therapeutic agents that are toxic to cancer stem cells or to therapeutic agents that target invasive, metastatic, or invasive and metastatic cancer cells.

29. The method of claim 16, wherein the reduced expression of genes in Table 2 indicates an increased likelihood that the tumor will be sensitive to therapeutic agents that are toxic to cancer cells that have undergone an epithelial-to-mesenchymal transition.

30. The method of claim 16, wherein the reduced expression of genes in Table 2 indicates an increased likelihood that the tumor will be sensitive to salinomycin.

31. A method of identifying therapeutic agents that target cancer stem cells or epithelial cancers that have undergone an epithelial to mesenchymal transition comprising screening candidate agents to identify those that increase the levels of expression of the genes in Table 2, wherein an increase in the expression of genes in Table 2 indicates that the candidate agent targets cancer stem cells or epithelial cancers that have undergone an epithelial to mesenchymal transition.

32. The method of claim 31, wherein any subset of genes in Table 2 is evaluated for its expression levels.

33. The method of claim 32, wherein the subset of genes is one for which a statistical test demonstrates that the genes in the subset are differentially expressed in populations treated with a cancer therapy at a level of significance less than 0.1, relative to an appropriate control population.

34. The method of claim 33, wherein the cancer therapy is selected from the group consisting of salinomycin treatment and paclitaxel treatment.

35. The method of claim 32, wherein the subset of genes comprises 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 2.

36. A method of identifying therapeutic agents that target cancer stem cells or epithelial cancers that have undergone an epithelial to mesenchymal transition comprising screening candidate agents to identify those that decrease the levels of expression of the genes in Table 1, wherein a decrease in the expression of genes in Table 1 indicates that the candidate agent targets cancer stem cells or epithelial cancers that have undergone an epithelial to mesenchymal transition.

37. The method of claim 36, wherein any subset of genes in Table 1 is evaluated for its expression levels.

38. The method of claim 37, wherein the subset of genes whose expression is evaluated is one for which a statistical test demonstrates that the genes in the subset are differentially expressed in populations treated with a cancer therapy at a level of significance less than 0.1, relative to an appropriate control population.

39. The method of claim 38, wherein the cancer therapy is selected from the group consisting of salinomycin treatment and paclitaxel treatment.

40. The method of claim 37, wherein the subset of genes comprises 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 1.

41. A method of predicting the likelihood that a patient's epithelial cancer will respond to therapy, following surgical removal of the primary tumor, comprising determining the expression level in cancer of genes in Table 1, wherein the overexpression of genes in Table 1 indicates an increased likelihood that the tumor will be sensitive to therapy with salinomycin or other CSS agents.

42. A method of predicting the likelihood that a patient's epithelial cancer will respond to therapy, following surgical removal of the primary tumor, comprising determining the expression level in cancer of genes in Table 1, wherein the overexpression of genes in Table 1 indicates an increased likelihood that the tumor will be resistant to standard-of-care therapy.

43. The method of claim 42 wherein the standard-of-care therapy is paclitaxel.

44. The method of claim 41, wherein any subset of genes in Table 1 is evaluated for its expression levels.

45. The method of claim 44, wherein the subset of the genes whose expression is evaluated is one for which a statistical test demonstrates that the genes in the subset are differentially expressed in populations treated with a cancer therapy at a level of significance less than 0.1, relative to an appropriate control population.

46. The method of claim 45, wherein the cancer therapy is selected from the group consisting of salinomycin treatment and paclitaxel treatment.

47. The method of claim 42, wherein the subset of genes comprises 2, 3, 4, 5, 6, 7, 8, 9, 10, 11, 12, 13, 14, 15, 16, 17, 18, 19, 20, 21, 22, 23, 24, 25, 26, 27, 28, 29, or 30 of the genes in Table 1.

48. The method of claim 1, further comprising summarizing the data obtained by the determination of said gene expression levels.

49. The method of claim 48, wherein said summarizing includes prediction of the likelihood of long term survival of said patient without recurrence of the cancer following surgical removal of the primary tumor.

50. The method of claim 48, wherein said summarizing includes recommendation for a treatment modality of said patient.

51. A kit comprising in one or more containers, at least one detectably labeled reagent that specifically recognizes one or more of the genes in Table 1 or Table 2.

52. The kit of claim 51, wherein the level of expression of the one or more genes in Table 1 or Table 2 in cancer is determined.

53. The kit of claim 51, wherein the kit is used to generate a biomarker profile of an epithelial cancer.

54. The kit of claim 51, wherein the kit further comprises at least one pharmaceutical excipient, diluents, adjuvant, or any combination thereof.

55. The method of claim 1, wherein the RNA expression levels are indirectly evaluated by determining protein expression levels of the corresponding gene products.

56. The method of claim 55, wherein the RNA expression levels are indirectly evaluated by determining chromatin states of the corresponding genes.

57. The method of claim 55 wherein said RNA is isolated from a fixed, wax-embedded breast cancer tissue specimen of said patient.

58. The method of claim 55, wherein said RNA is fragmented RNA.

59. The method of claim 55, wherein said RNA is isolated from a fine needle biopsy sample.

60. The method of claim 1, wherein the cancer is an epithelial cancer.

61. The method of claim 1, wherein the cancer is a lung, breast, prostate, gastric, colon, pancreatic, brain, or melanoma cancer.
